(19) United States
(12) Patent Application Publication
(10) Pub. No.: US 2011/0028740 A1
(43) Pub. Date: Feb. 3, 2011

(54) METHOD FOR STARTING A GAS PHASE OXIDATION REACTOR THAT CONTAINS A CATALYTICALLY ACTIVE SILVER-VANADIUM OXIDE BRONZE

(75) Inventors: Cornelia Dobner, Ludwigshafen (DE); Ulf Dietrich, Grunstadt (DE); Thomas Mackewitz, Singapore (SG); Sebastian Storck, Mannheim (DE); Hagen Wilmer, Ludwigshafen (DE); Frank Rosowski, Mannheim (DE); Andreas Tenten, Neustadt/Weinstrasse (DE)

Correspondence Address: CONNOLLY BOVE LODGE & HUTZ LLP, P.O. BOX 2207, WILMINGTON, DE, 19899, US

(73) Assignee: BASF SE, Ludwigshafen (DE)

(21) Appl. No.: 12/936,648

(22) PCT Filed: Apr. 7, 2009
(86) PCT No.: PCT/EP09/54170

§ 371 Date: Oct. 6, 2010

(30) Foreign Application Priority Data
Apr. 7, 2008 (EP) 08154168.2

(51) Int. Cl. C07D 307/89; C07D 307/36; C07C 51/347; C07C 63/00; C07C 63/04

(52) U.S. Cl. 549/247; 549/262; 562/400; 562/480; 562/493

(57) ABSTRACT

What is described is a method for starting a gas phase oxidation reactor that contains a bed of a first catalyst whose active material comprises a catalytically active silver-vanadium oxide bronze, and at least one bed of a second catalyst whose catalytically active material comprises vanadium pentoxide and titanium dioxide, and whose temperature is controllable by means of a heat transfer medium. In the operating state, a gas stream which comprises a loading cₒₚ of a hydrocarbon and molecular oxygen is passed through the reactor over the bed of the first and second catalyst at a temperature Tₒₚ of the heat transfer medium. For the startup, a) a gas stream is passed through the reactor with a starting loading c₀ which is less than cₒₚ, and at a starting temperature T₀ of the heat transfer medium which is less than Tₒₚ, and b) the temperature of the heat transfer medium is brought to Tₒₚ and the loading of the gas stream to cₒₚ. The process combines a short startup time without exceedance of emissions or quality requirements, long catalyst lifetime, high yield and low formation of by-products.

A multitude of carboxylic acids and/or carboxylic anhydrides, for example benzoic acid, maleic anhydride, phthalic anhydride, isophthalic acid, terephthalic acid or pyromellitic anhydride, is prepared industrially by the catalytic gas phase oxidation of aromatic hydrocarbons, such as benzene, the xylenes, naphthalene, toluene or durene, in fixed bed reactors.

To this end, a gas stream which comprises molecular oxygen and the hydrocarbon to be oxidized is generally passed through a multitude of tubes arranged in a reactor, in which a bed of at least one catalyst is disposed. For temperature regulation, the tubes are surrounded by a heat carrier medium, for example a salt melt. In spite of this thermostating, so-called “hotspots” are formed in the catalyst bed, in which there is a higher temperature than in the remainder of the catalyst bed. These “hotspots” cause side reactions, such as the total combustion of the starting material, or lead to the formation of undesired by-products which can be removed from the reaction product only with great difficulty, if at all, for example to the formation of phthalide or benzoic acid in the preparation of phthalic anhydride (PA) from o-xylene.

To attenuate these hotspots, there has been a move in industry to arranging catalysts of different activity layer by layer in the catalyst bed, the less active catalyst generally being arranged in the fixed bed such that the reaction gas mixture comes into contact with it first, i.e. it is present in the bed toward the gas inlet, whereas the more active catalyst is present toward the gas outlet from the catalyst bed (DE-A 25 46 268, EP 286 448, DE 29 48 163, EP 163 231).

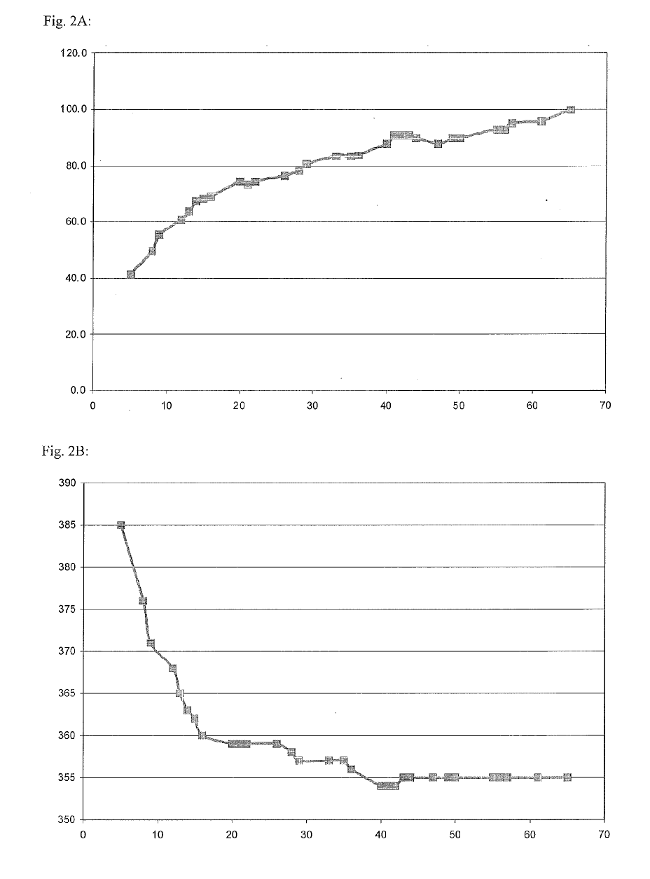

To put the reactor into operation, or to “start it up”, the catalyst bed is typically brought by external heating to a temperature which is above the later operating temperature. As soon as the oxidation reaction commences, the reaction temperature is maintained by the marked exothermicity of the conversion and the external heating is reduced and finally switched off. The formation of a marked hotspot, however, prevents a rapid startup phase (runup phase), since, from a particular hotspot temperature, the catalyst can be damaged irreversibly. Therefore, the loading of the gas stream with the hydrocarbon to be oxidized is increased hi small steps and it is necessary to undertake very careful monitoring. Typical plots of the loading of the gas stream and of the salt bath temperature on startup of a reactor for oxidation of o-xylem to phthalic anhydride are shown in the appended FIG. 2A and FIG. 2B.

WO 98/00778 discloses that the addition of temporary activity attenuators can lead to a shortening of the startup phase.

In spite of the proposed improvements mentioned above, long startup times of from 2 to 8 weeks or longer have been required to date. “Startup time” describes the time which is needed to bring the supply of the hydrocarbon to the desired end loading, i.e. to bring the oxidation to a steady state, without irreversibly damaging the catalyst. In this context, it should be ensured in particular that the hotspot does not exceed a certain critical value, since the selectivity and the lifetime of the catalyst are otherwise greatly impaired.

On the other hand, the salt bath temperature on startup cannot be selected at as low a level as desired, since increased contents of unconverted hydrocarbon and/or underoxidation products otherwise occur in the reaction product, which can lead to exceedance of emissions or quality requirements.

In the case of the industrially important oxidation of o-xylene to phthalic anhydride, the end loading is, for example, 80 g of o-xylene/m³ (STP) of air or more. The catalysts based on vanadium oxide and titanium dioxide which have been used to date are started up at temperatures of 360-400° C., preferably 370-390° C. This ensures that the remaining amount of o-xylene and the content of the phthalide underoxidation product are within the emissions and quality requirements. In the course of the forming phase which then follows, the salt bath temperature is lowered (to typically 350° C.), and the loading can be increased to target load in parallel.

WO 00/27753, WO 01/85337 and WO 2005/012216 describe silver-vanadium oxide bronzes which selectively catalyze the partial oxidation of aromatic hydrocarbons. The silver-vanadium oxide bronzes are appropriately used in combination with catalysts based on vanadium oxide and titanium dioxide. A gas stream which comprises a hydrocarbon and molecular oxygen is passed successively over a bed of a first, i.e. upstream, catalyst whose catalytically active material comprises the catalytically active silver-vanadium oxide bronze, and at least one bed of a second, i.e. downstream, catalyst whose catalytically active material comprises vanadium pentoxide and titanium dioxide. The hydrocarbon is converted first over the bed of the first catalyst, with partial conversion to a gaseous reaction mixture. The conversion is then completed over the bed of the second catalyst.

The prior art does not hold ready any method for starting a gas phase oxidation reactor that contains a catalytically active silver-vanadium pentoxide bronze.

Compared to known catalysts based on vanadium pentoxide and titanium dioxide, silver-vanadium oxide bronzes are very active and can therefore be started up only at low temperatures. At higher temperatures, exceedance of the permissible hotspot temperature in the silver-vanadium oxide bronze bed and hence damage to the catalyst would be the consequence.

At these low startup temperatures, however, an increase in the hydrocarbon loading leads to impermissible contents of unconverted hydrocarbon and/or underoxidation products since the downstream catalyst bed does not form sufficiently rapidly at these low temperatures.

It was therefore an object of the invention to specify a process for starting up a gas phase oxidation reactor, which combines a short startup time without exceedance of emissions and quality requirements, long catalyst lifetime, high yield and low formation of by-products.

The object is achieved by a method for starting a gas phase oxidation reactor that contains a bed of a first catalyst whose active material comprises a catalytically active silver-vanadium oxide bronze, and at least one bed of a second catalyst whose catalytically active material comprises vanadium pentoxide and titanium dioxide, and whose temperature is controllable by means of a heat transfer medium, to an operating state in which a gas stream which comprises a loading cₒₚ, of a hydrocarbon and molecular oxygen is passed through the reactor over the bed of the first and second catalyst at a temperature Tₒₚ of the heat transfer medium, wherein
a) a gas stream is passed through the reactor with a starting loading c₀ which is less than cₒₚ, and at a starting temperature T₀ of the heat transfer medium which is less than Tₒₚ,
b) the temperature of the heat transfer medium is brought to Tₒₚ, and the loading of the gas stream to cₒₚ.

The operating state is considered to be the essentially steady state of the reactor in productive operation after the startup phase has ended. The operating state is characterized by an essentially constant temperature of the heat transfer medium Tₒₚ and an essentially constant loading of the gas stream cₒₚ. To compensate for declining catalyst activity, in the operating state, the temperature of the heat transfer medium can, however, be increased over a long period (less than 2.5° C./year).

In the process according to the invention, the gas stream is passed successively over a bed of the first, i.e. upstream, catalyst and at least one bed of a second, i.c. downstream, catalyst. The hydrocarbon is first converted over the bed of the first catalyst, whose active material comprises a catalytically active silver-vanadium oxide bronze, with partial conversion to a gaseous reaction mixture. The resulting reaction mixture is then contacted with at least one second catalyst whose catalytically active material comprises vanadium pentoxide and titanium dioxide.

Reaction products which are prepared by gas phase oxidation in the reactor are, for example, benzoic acid, maleic anhydride, phthalic anhydride, isophthalic acid, terephthalic acid or pyromellitic anhydride. The hydrocarbons used are in particular aromatic hydrocarbons, such as benzene, alkylated benzenes, such as toluene, the xylenes, durene, or naphthalene. A preferred embodiment of the invention relates to the oxidation of o-xylene to phthalic anhydride.

In a suitable embodiment of the process, the procedure in step b) is as follows
b1) at essentially constant loading of the gas stream, the temperature of the heat transfer medium is increased, and then
b2) at essentially constant temperature of the heat transfer medium, the loading of the gas stream is increased,
   and steps b1) and b2) are, if appropriate, repeated once or more than once until the temperature of the heat transfer medium is equal to Tₒₚ and the loading of the gas stream is equal to cₒₚ.

In a preferred embodiment of the process, the procedure in step b) is as follows
b1) at essentially constant loading of the gas stream, the temperature of the heat transfer medium is increased to Tₒₚ, and then
b2) at essentially constant temperature of the heat transfer medium, the loading of the gas stream is increased to cₒₚ.

In general, the increase in the loading of the gas stream is regulated such that the content of unconverted hydrocarbon and/or of at least one underoxidation product in the reaction product does not exceed a predetermined limit.

The content of unconverted hydrocarbon and/or underoxidation products can be determined by, at about 23° C., condensing all components of the reaction product which can be condensed at this temperature, and analyzing the condensate by means of gas chromatography in a suitable solvent, such as acetone. The content of unconverted hydrocarbon and/or underoxidation products is, in the present context, based on the total weight of the condensed reaction product.

Unconverted hydrocarbons, such as o-xylene, are typically not washed out or condensed in the workup apparatus connected downstream of the reactor; they are therefore emitted and lead to undesired environmental pollution. For this reason, the content of unconverted hydrocarbon in the reaction product should not exceed a predetermined limit.

Underoxidation products are considered to be molecules which possess the same number of hydrocarbons as the desired oxidation product but possess a lower oxidation state than the desired oxidation product and can be oxidized further to the desired oxidation product. Underoxidation products generally can be removed from the desired oxidation product only with a disproportionately high level of complexity, if at all. Elevated contents of underoxidation products reduce the product quality.

Underoxidation products of phthalic anhydride are especially o-tolylaldehyde, o-toluic acid and phthalide. Preference is given to concentrating on phthalide as an underoxidation product, since the phthalide concentration is considered to be a guide parameter for colored high boilers. Elevated phthalide values lead to off-spec products, since the permissible color number is then exceeded.

In the case of oxidation of o-xylene to phthalic anhydride, the concentration of unconverted o-xylene is preferably not more than 0.1% by weight in the reaction product. The concentration of phthalide formed is preferably not more than 0.20% by weight in the reaction product.

In the case of operation of a phthalic anhydride reactor with a postreactor, higher limits are permissible for the concentrations of unconverted o-xylene and phthalide. Postreactors are described, for example, in DE 20 05 969, DE 198 07 018 and U.S. Pat. No. 5,969,160. For instance, in the case of operation with a postreactor, the concentration of unconverted o-xylene are preferably not more than 3% by weight. The concentration of phthalide is preferably not more than 1% by weight.

From a particular loading of the gas stream, a hotspot, i.e. a temperature maximum, forms in the bed of the second catalyst. The further increase in the loading of the gas stream is preferably regulated such that the temperature at the hotspot in the bed of the second catalyst does not exceed a predetermined limit. For instance, a temperature of 440° C. is preferably not exceeded, since the selectivity and the lifetime of the catalyst are otherwise significantly impaired. On the other hand, the temperature should not go below 400° C., in order that the forming of the second catalyst proceeds.

In a suitable embodiment, the starting loading is at least 30 g/m³ (STP) lower than cₒₚ. In the ease of o-xylene, the minimum loading of the gas stream is generally 30 g of o-xylene/m³ (STP), because the homogeneous spraying of the o-xylene metered in liquid form is ensured only from this amount.

In a suitable embodiment, the starting temperature is at least 8° C. lower than Tₒₚ.

In a suitable embodiment, the loading cₒₚ is from 60 to 110 g/m³ (STP), preferably from 80 to 100 g/m³ (STP).

In a suitable embodiment, the temperature Tₒₚ is from 340 to 365° C., preferably from 350 to 360° C.

In a suitable embodiment, in step b1), the temperature is increased at a rate of from 0.5 to 5° C./day.

In a suitable embodiment, in step b2), the loading is increased at a rate of from 0.5 to 10 g/m³ (STP) day.

Silver-vanadium oxide bronzes are understood to mean silver-vanadium oxide compounds with an atomic Ag:V ratio of less than 1. They are generally semiconductive or metallically conductive, oxidic solids which crystallize preferentially in sheet or tunnel structures, the vanadium in the [V₂O₅] host lattice being present at least partly reduced to V (IV). Suitable first catalysts whose active material comprises a catalytically active silver-vanadium oxide bronze are described in WO 00/27753, WO 01/85337 and WO 2005/012216. The silver-vanadium oxide bronze is obtainable by thermal treatment of suitable multimetal oxides. The thermal conversion of the multimetal oxides to silver-vanadium oxide bronzes proceeds via a series of reduction and oxidation reactions whose details are as yet not understood. In practice, the multimetal oxide layer is applied to an inert support to obtain a so-called precatalyst. The precatalyst can be converted to the active catalyst in situ in the gas phase oxidation reactor under the conditions of the oxidation of aromatic hydrocarbons. Alternatively and preferably, the precatalyst is converted to the active catalyst ex situ before the introduction into the gas phase oxidation reactor, as described in WO 2007/071749.

In general, the multimetal oxide has the general formula I:

Ag_{a-c}Q_{b}M_{c}V₂O_{d}*eH₂O I

in which
a is from 0.3 to 1.9, preferably from 0.5 to 1.0 and more preferably from 0.6 to 0.9;
Q is an element selected from P, As, Sb and/or Bi,
b is from 0 to 0.3, preferably from 0 to 0.1,
M is at least one metal selected from alkali metals and alkaline earth metals, Bi, Tl, Cu, Zn, Cd, Pb, Cr, Au, Al, Fe, Co, Ni, Mo, Nb, Ce, W, Mn, Ta, Pd, Pt, Ru and/or Rh, preferably Nb, Ce, W, Mn and Ta, especially Ce and Mn, of which Ce is the most preferred,
c is from 0 to 0.5, preferably from 0.005 to 0.2, especially from 0.01 to 0.1; with the proviso that (a-c) is ≧0.1,
d is a number which is determined by the valency and frequency of the elements in the formula I other than oxygen, and
e is from 0 to 20, preferably from 0 to 5.

Preferably, the silver-vanadium oxide bronze is present in a crystal structure whose powder X-ray diagram is characterized by reflections at the interplanar spacings d of 4.85±0.4, 3.24±0.4, 2.92±0.4, 2.78±0.04, 2.72±0.04, 2.55±0.04, 2.43±0.04, 1.95±0.04 and 1.80±0.04 Å. In this application, the X-ray reflections are reported in the form of the interplanar spacings d[Å] which are independent of the wavelength of the X-radiation used and can be calculated from the diffraction angle measured by means of the Bragg equation.

To prepare the multimetal oxides, a suspension of vanadium pentoxide (V₂O₅) is generally heated with the solution of a silver compound and if appropriate a solution of a compound of the metal component M and of a compound of Q. The solvent used for this reaction is preferably water. The silver salt used is preferably silver nitrate; the use of other soluble silver salts, for example silver acetate, silver perchlorate or silver fluoride, is likewise possible.

The salts of the metal component M selected are generally those which are soluble in the solvent used. When water is used as the solvent in the preparation of the inventive multimetal oxides, it is possible, for example, to use the perchlorates or carboxylates, especially the acetates, of the metal component M. Preference is given to using the nitrates of the metal component M in question.

According to the desired chemical composition of the multimetal oxide of the formula I, it is prepared by reacting the amounts of V₂O₅, silver compound and the compound of the metal component M which arise from a and c of formula I with one another. The multimetal oxide thus formed can be isolated from the reaction mixture and stored until further use. Particularly advantageously, the isolation of the resulting multimetal oxide suspension is carried out by means of spray drying. The spray-dried powder is then applied to an inert support.

Catalysts based on vanadium pentoxide and titanium dioxide comprise, as well as titanium dioxide (in the form of its anatase polymorph), vanadium pentoxide. In addition, it is possible for small amounts of other oxidic compounds which, as promoters, influence the activity and selectivity of the catalyst to be present. Activity-reducing and selectivity-increasing promoters used are generally alkali metals, such as cesium, lithium, potassium and rubidium, and especially cesium. Activity-increasing additives used are generally phosphorus compounds. The catalysts based on vanadium oxide and titanium dioxide may also comprise antimony compounds. Typical catalysts based on vanadium oxide and titanium dioxide and their preparation are described in DE 198 23 262.

In general, the catalytically active material of the second catalyst comprises from 1 to 40% by weight of vanadium oxide, calculated as V₂O₅, from 60 to 99% by weight of titanium dioxide, calculated as TiO₂, up to 1% by weight of a cesium compound, calculated as Cs, up to 1% by weight of a phosphorus compound, calculated as P, and up to 10% by weight of antimony oxide, calculated as Sb₂O₃.

Advantageously, the bed of the second catalyst comprises at least two layers of catalysts whose catalytically active material has a different Cs content, the Cs content decreasing in flow direction of the gas stream.

The components are used in the form of their oxides or in the form of compounds which are converted to oxides in the course of heating or in the course of heating in the presence of oxygen. The vanadium components used may be vanadium oxides or vanadium compounds which are converted to vanadium oxides in the course of heating, individually or in the form of mixtures thereof. Preference is given to using V₂O₅ or NH₄ VO₃. It is also possible to use a reducing agent, such as formic acid or oxalic acid, in order to reduce the vanadium (V) compound at least partly to vanadium (IV). Suitable promoter (precursor) compounds are the corresponding oxides or compounds which are converted to oxides after heating, such as sulfates, nitrates, carbonates. Suitable examples are Na₂CO₃, K₂O, Cs₂O, Cs₂CO₃, Cs₂SO₄, P₂O₅, (NH₄)₂HPO₄, Sb₂O₃.

To form the active material, an aqueous slurry of the compound of the vanadium component, of the titanium dioxide and of promoter (precursor) compounds is generally prepared in suitable amounts and the slurry is stirred until sufficient homogenization is achieved. The slurry can then be spray-dried or be used as such for coating.

The catalysts used in the process according to the invention are generally coated catalysts in which the catalytically active material is applied in coating form on an inert support. The layer thickness of the catalytically active material is generally from 0.02 to 0.2 mm, preferably from 0.05 to 0.1 mm. In general, the catalysts have an active material layer of essentially homogeneous chemical composition applied in coating form.

The inert support materials used may be virtually all known support materials, for example quartz (SiO₂), porcelain, magnesium oxide, tin dioxide, silicon carbide, rutile, alumina (Al₂O₃), aluminum silicate, steatite (magnesium silicate), zirconium silicate, cerium silicate or mixtures of these support materials. The support material is generally nonporous. Advantageous support materials which should be emphasized are especially steatite and silicon carbide. The shape of the support material is generally uncritical. For example, catalyst supports can be used in the form of spheres, rings, tablets, spirals, tubes, extrudates or spall. The dimensions of these catalyst supports correspond to those of catalyst supports typically used to prepare coated catalysts for the gas phase partial oxidation of aromatic hydrocarbons. Preference is given to using steatite in the form of spheres with a diameter of from 3 to 6 mm or of rings with an external diameter of from 5 to 9 mm and a length of from 4 to 7 mm.

The active material layer can be applied to the support by any methods known per se, for example by spraying on solutions or suspensions in a coating drum or coating with a solution or suspension in a fluidized bed. In this case, organic binders, preferably copolymers, advantageously in the form of an aqueous dispersion, of vinyl acetate/vinyl laurate, vinyl acetate/acrylate, styrene/acrylate, vinyl acetate/maleate, vinyl acetate/ethylene and hydroxyethylcellulose can be added to the catalytically active material, the amounts of hinder used advantageously being from 3 to 20% by weight, based on the solids content of the solution of the active material constituents. The binders applied burn off within a short time after the catalyst has been introduced and the reactor has been put into operation. The addition of binder additionally has the advantage that the active material adheres efficiently on the support, such that transport and introduction of the catalyst are facilitated.

The temperature of the gas phase oxidation reactor is controllable by means of a heat transfer medium. In general, the temperatures of the bed of the first catalyst and of the bed of the second catalyst are controllable by a common heat transfer medium, for example by means of a single salt bath circuit. The reactor used is preferably a tubular reactor cooled by a salt bath. This reactor comprises a tube bundle around which a heat carrier medium in the form of a salt bath flows. The individual catalyst-filled tubes end in an upper tube plate and a lower tube plate. The reaction gas is generally passed through the tubes from the top downward, i.e. in the direction of gravity; however, a reverse flow direction is also conceivable. On the outside of the reactor are disposed, spaced apart from one another, ring channels through which the heat carrier medium is drawn off from the reactor and fed back to the reactor after passing through a circulation pump. A substream of the circulated heat carrier medium is passed through a cooler in which, for example, saturated steam is produced. In the interior of the reactor, guide plates may be present in a customary manner in order to impart a radial flow component to the heat carrier medium in the region of the tube bundle.

In the present context “temperature of the heat transfer medium” is considered to be the lowest temperature of the heat transfer medium in the area of the reactor, and is generally the temperature in the feed of the salt melt into the reactor.

The catalysts are filled into the reaction tubes of the tubular reactor. The reaction gas is passed through the catalyst bed thus prepared.

The reaction gas fed to the reactor is generally obtained by mixing a molecular oxygen-comprising gas which, apart from oxygen, may also comprise suitable reaction moderators and/or diluents, such as steam, carbon dioxide and/or nitrogen, with the aromatic hydrocarbon to be oxidized, and the molecular oxygen-comprising gas may comprise generally from 1 to 100% by volume, preferably from 2 to 50% by volume and more preferably from 10 to 30% by volume of oxygen, from 0 to 30% by volume and preferably from 0 to 20% by volume of steam, and from 0 to 50% by volume and preferably from 0 to 1% by volume of carbon dioxide, remainder nitrogen. Particularly advantageously, the molecular oxygen-comprising gas used is air.

The invention is illustrated in detail by the appended drawings and the examples which follow.

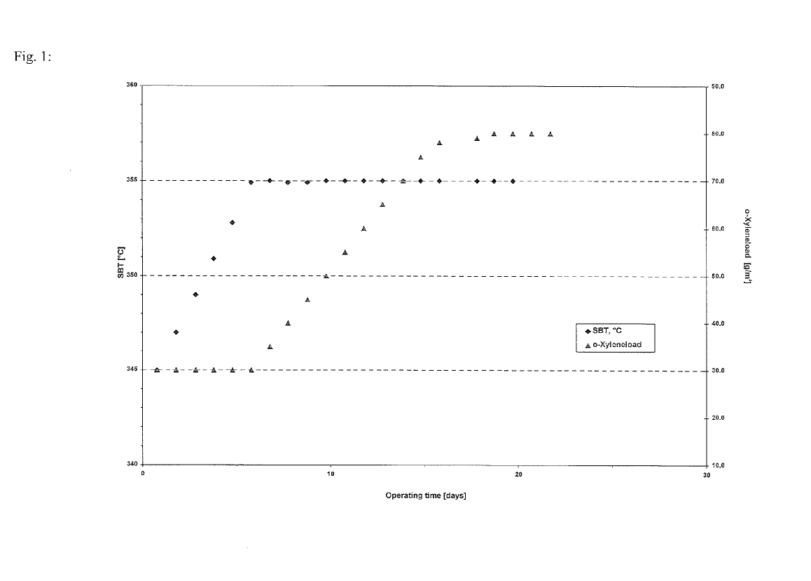

FIG. 1 shows the salt bath temperature and the loading of the gas stream plotted against time in one embodiment of the process according to the invention for oxidizing o-xylene to phthalic anhydride.

FIG. 2A shows the loading of the gas stream (g/m³ (STP)) plotted against time (days) and FIG. 2B the salt bath temperature (° C.) on startup of a reactor for oxidizing o-xylene to phthalic anhydride, said reactor comprising exclusively catalysts based on vanadium pentoxide and titanium dioxide, plotted against time (days).

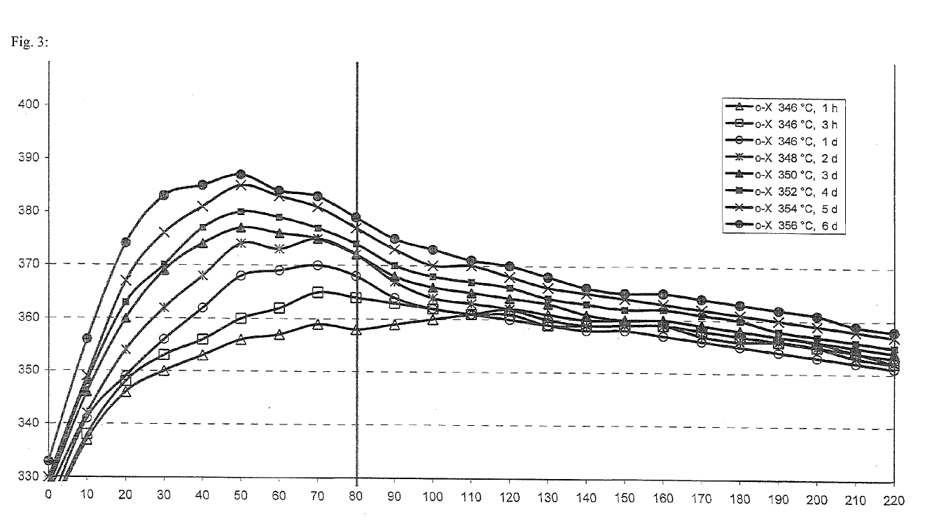

FIG. 3 shows a typical local temperature profile (° C.) in a reaction tube (cm from the reactor inlet) at constant loading of 30 g/m³ (STP), while the salt bath temperature is increased from 346° C. to 356° C. within 6 days.

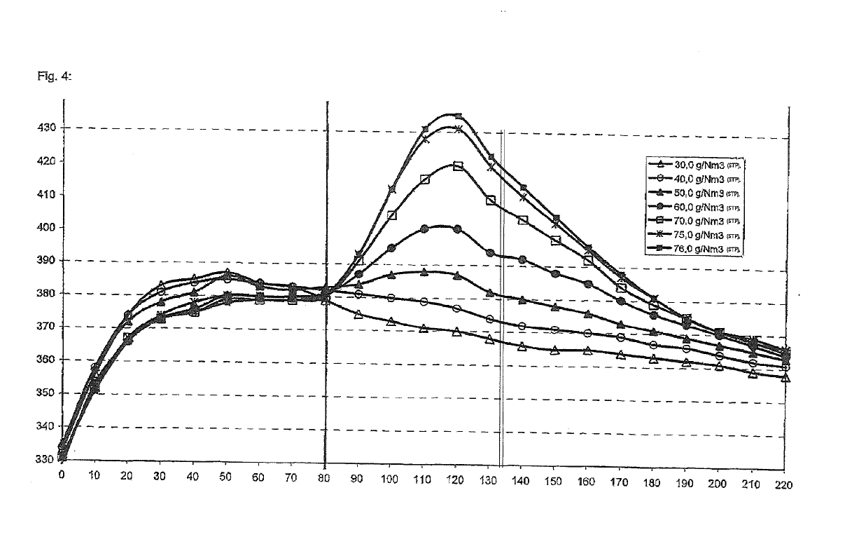

FIG. 4 shows a typical local temperature profile (° C.) in a reaction tube (cm from the reactor inlet) at constant salt bath temperature of 356° C., while the loading is increased from 30 g/m³ (STP) to 76 g/m³ (STP).

### EXAMPLE

A reactor with 100 tubes of length 360 cm, which was surrounded by a salt bath, was used. The tubes were charged up to a fill height of 240 cm with a catalyst based on vanadium pentoxide and titanium dioxide. The catalyst comprised an active material of composition 5.75% by weight of V₂O₅, 1.58% by weight of Sb₂O₃, 0.11% by weight of P, 0.41% by weight of Cs, 0.027% by weight of K, remainder TiO₂, which was applied to steatite supports in the form of hollow cylinders (8×6×5 mm); the active material content was 9% by weight. Subsequently, up to a fill height of 320 cm, catalyst whose active material comprised a silver-vanadium oxide bronze of composition Ag_{0.68}V₂O₅ was introduced (for its preparation see WO 00/27753).

The reactor also comprised a thermal tube which allowed temperature measurement axially along the catalyst beds. For this purpose, the thermal tube, in addition to the fixed catalyst bed, comprised a thermowell charged only with a temperature sensor and conducted along the center of the thermal tube.

Within 20 h, the salt bath was heated to 200° C., in the course of which 2 m³/h/tube of air were passed through the tubes. Subsequently, the salt bath temperature was increased to 380° C. without passing air through within 25 h. 3.81 m³ (STP)/h/tube of air were then passed through the tubes at salt bath temperature 380° C. for 30 min In the course of this, the salt bath reactor cools. On attainment of a temperature of 346° C., 30 g/m³ (STP) of o-xylene were metered into the air stream. Owing to the exothermicity on startup of the oxidation reaction, the temperature of the reactor did not fall any further. With an unchanged loading of 30 g/m³ (STP) of o-xylene, the salt bath temperature was increased from the 2nd day by 2° C./day until, after 5 days, a salt bath temperature of 356° C. had been attained. A typical local temperature profile (° C.) in a reaction tube (cm from the reactor inlet) is shown in FIG. 3. It can be seen that a hotspot forms in the bed of the first catalyst.

Subsequently, the loading was increased at 5 g/m³ (STP).day to 76 g/m³ (STP). A typical local temperature profile (° C.) in a reaction tube (cm from the reactor inlet) is shown in FIG. 4. It can be seen that a hotspot forms in the bed of the second catalyst; the critical hotspot temperature in the bed of the first catalyst is not exceeded. Nevertheless, the hotspot in the bed of the second catalyst reaches a temperature sufficient for the formation of the catalyst.

1.-14. (canceled)

15. A method for starting a gas phase oxidation reactor that contains a bed of a first catalyst whose active material comprises a catalytically active silver-vanadium oxide bronze, and at least one bed of a second catalyst whose catalytically active material comprises vanadium pentoxide and titanium dioxide, and whose temperature is controllable by means of a heat transfer medium, to an operating state in which a gas stream which comprises a loading cₒₚ of a hydrocarbon and molecular oxygen is passed through the reactor over the bed of the first and second catalyst at a temperature Tₒₚ of the heat transfer medium, wherein
a) a gas stream is passed through the reactor with a starting loading c₀ which is less than cₒₚ, and at a starting temperature T₀ of the heat transfer medium which is less than Tₒₚ,
b) the temperature of the heat transfer medium is brought to Tₒₚ and the loading of the gas stream to cₒₚ.

16. The method according to claim 15, wherein, in step b),
b1) at essentially constant loading of the gas stream, the temperature of the heat transfer medium is increased, and then
b2) at essentially constant temperature of the heat transfer medium, the loading of the gas stream is increased,
and steps b1) and b2) are, if appropriate, repeated once or more than once until the temperature of the heat transfer medium is equal to Tₒₚ and the loading of the gas stream is equal to cₒₚ.

17. The method according to claim 16, wherein
b1) at essentially constant loading of the gas stream, the temperature of the heat transfer medium is increased to Tₒₚ, and then
b2) at essentially constant temperature of the heat transfer medium, the loading of the gas stream is increased to cₒₚ.

18. The method according to claim 16, wherein the increase in the loading of the gas stream is regulated such that the content of unconverted hydrocarbon and/or of at least one underoxidation product in the reaction product does not exceed a predetermined limit.

19. The method according to claim 18, wherein the hydrocarbon is o-xylene and is oxidized to phthalic anhydride, and the underoxidation product is phthalide.

20. The method according to claim 15, wherein the increase in the loading of the gas stream is regulated such that the temperature at the hotspot in the bed of the second catalyst does not exceed a predetermined limit.

21. The method according to claim 15, wherein the starting loading is at least 30 g/m³ (STP) lower than cₒₚ.

22. The method according to claim 15, wherein the starting temperature is at least 8° C. lower than Tₒₚ.

23. The method according to claim 15, the loading cₒₚ is from 60 to 110 g/m³ (STP).

24. The method according to claim 15, wherein the temperature Tₒₚ is from 340 to 365° C.

25. The method according to claim 16, wherein, in step b1), the temperature is increased at a rate of from 0.5 to 5° C./day.

26. The method according to claim 16, wherein, in step b2), the loading is increased at a rate of from 0.5 to 10 g/m³ (STP).day.

27. The method according to claim 15, wherein the silver-vanadium oxide bronze is obtainable from a multimetal oxide of the general formula I
Ag_{a-c}Q_{b}M_{c}V₂O_{d}*eH₂O,
in which
a is from 0.3 to 1.9,
Q is an element selected from P, As, Sb and/or Bi,
b is from 0 to 0.3,
M is at least one metal selected from alkali metals and alkaline earth metals, Bi, Tl, Cu, Zn, Cd, Pb, Cr, Au, Al, Fe, Co, Ni, Mo, Nb, Ce, W, Mn, Ta, Pd, Pt, Ru and/or Rh,
c is from 0 to 0.5, with the proviso that (a-c) is ≧0.1,
d is a number which is determined by the valency and frequency of the elements in the formula I other than oxygen, and
e is from 0 to 20.

28. The method according to claim 15, wherein the silver-vanadium oxide bronze is present in a crystal structure whose powder X-ray diagram is characterized by reflections at the interplanar spacings d 4.85±0.4, 3.24±0.4, 2.92±0.4, 2.78±0.04, 2.72±0.04, 2.55±0.04, 2.43±0.04, 1.95±0.04 and 1.80±0.04 Å.
